(19) United States
(12) Patent Application Publication
(10) Pub. No.: US 2004/0241246 A1
(43) Pub. Date: Dec. 2, 2004

(54) Fluid absorbing adhesive paste

(76) Inventor: Roger D. A. Lipman, Turnhout (BE)

Correspondence Address: Heidi A. Boehlefeld, Renner, Otto, Boisselle & Sklar, LLP, Nineteenth Floor, 1621 Euclid Avenue, Cleveland, OH, 44115-2191, US

(21) Appl. No.: 10/849,645

(22) Filed: May 19, 2004

Related U.S. Application Data
(60) Provisional application No. 60/472,272, filed on May 20, 2003.

(51) Int. Cl. A61K 9/70; A61F 13/00; A61K 9/14

(52) U.S. Cl. 424/486; 424/449

(57) ABSTRACT

The present invention relates to an adhesive paste applicable to skin in paste form comprising a mixture of: (a) a continuous phase comprising at least one styrene-containing thermoplastic elastomer, at least one compatible liquid rubber, polyisobutylene, and oil; and (b) a discontinuous phase comprising at least one water soluble and/or water swellable polymer; wherein the adhesive paste contains about 25% to about 45% by weight, based on the total weight of the adhesive paste, of the oil.

This application claims priority to provisional application Ser. No. 60/472,272 filed on May 20, 2003, the content of which is hereby incorporated by reference in its entirety.

### TECHNICAL FIELD

The present invention relates to a fluid absorbing adhesive paste composition for use in wound management, e.g., on mammalian skin surfaces. In particular, the present invention relates to a fluid absorbing, pressure-sensitive adhesive paste composition for use in, e.g., skin resurfacing procedures.

### BACKGROUND

Skin resurfacing techniques include use of lasers, dermabrasion and chemical peels with alpha-hydroxy acids, phenol or trichloroacetic acid. These techniques, and particularly laser techniques, require extensive follow-up treatment to achieve the desired goal of skin resurfacing. The incidences of cosmetic surgery are increasing. It is believed that at least 300,000 such procedures are carried out each year in North America and Europe.

Despite the large numbers of such procedures, there remains a need for materials, methods and systems for treating and dressing wounds such as laser wounds. It is desirable to accelerate the healing and to restore the functional barrier property of the skin as rapidly as possible. Skin resurfacing procedures result in predictable post-operative sequelae including facial edema, wound exudate and erythema. In addition, there may be pain, pruritis, hyperpigmentation, milia formation and acne. It is desirable to address all of these consequences of wounding and aspects of healing. Thus, dressings used should absorb wound exudate sufficiently to minimize dressing changes, and should minimize incidence of wound infection, as well as reducing pain and pruritis, as well as contributing to a cosmetically acceptable result.

One of the elements needed for such treatment is a moldable adhesive dressing including a moldable pressure-sensitive adhesive hydrocolloid composition capable of use on and adherence to, contoured, uneven surfaces such as a human face, particularly in the nasal area.

Pressure-sensitive adhesive hydrocolloid formulations are comprised of dispersions of fluid absorbent materials in pressure-sensitive adhesive matrices. Many such adhesives are known in medical fields where they find use as functional components of medical devices. In particular, hydrocolloid adhesives have been extensively utilized in the fields of ostomy care and wound care. In ostomy care, hydrocolloid adhesives are used as the barrier adhesive to hold the pouch in place on the peristomal skin, and to protect this skin from the excoriating effects of body waste. In wound care, hydrocolloid adhesives have been used as the basis of absorbent dressings for direct application to especially chronic wounds.

Hydrocolloid pressure-sensitive adhesive compositions are sometimes not very flexible or conformable, so that adhesion to movable and curved body parts often is difficult. This lack of flexibility can give rise to problems. For example, an ostomy patient with folded or scarred skin in the region of his stoma may have difficulty in adhering the hydrocolloid adhesive barrier of his pouch to the peristomal skin without getting a leak between the skin and the adhesive. Leaks would allow highly irritating and excoriating feces or urine to come into contact with the skin. In order to aid leak free adhesion of barriers to undulating skin surfaces, paste products are sometimes used against the skin to fill depressions caused for example by scar tissue or by obesity.

The prior art discloses products designed to overcome the shortcomings of inflexible hydrocolloid skin barriers. In the field of ostomy care, adjunct products such as ostomy paste are well known. Such products have been used for protecting and treating the skin contiguous to a stoma. The problem of fluid leakage is often aggravated where the skin around the stoma is irregular, or where folds of skin occur in this area. To obtain an improved seal the ostomate can apply a coating of paste in a ring around the stoma, allow the paste to dry, and then apply the ostomy pouch over the dried paste.

Some known pressure-sensitive adhesive hydrocolloid-containing compositions are squeezable from a tube. The problem with these compositions is that in order to have a viscosity low enough for the user to squeeze them from the tubes, they contain a solvent, usually an alcohol such as isopropanol or ethanol. Such solvents are undesirable, since they can sting the skin on application. Often the preparation can harden in the tube due to solvent evaporation and become difficult or impossible to apply.

Other hydrocolloid-containing adhesive compositions are overly sticky. Even though such compositions are said to be removable from the skin as an integrated unit, without leaving residues, these compositions suffer from the drawback of being very sticky, and difficult to use because of the tackiness.

Thus, the prior art products described suffer from one or more of several drawbacks. If tube-dispensable, they often contain alcohols in order to make them tube dispensable. If not tube-dispensable, they are formulated as very sticky, tacky, and difficult-to-handle products for direct application to the skin.

For these reasons, a need remains for a pressure-sensitive adhesive hydrocolloid composition that is dispensable from a tube, free of solvents, not overly sticky and yet is adherent to contoured surfaces such as the human face.

### SUMMARY

The present invention relates to a fluid absorbing adhesive paste composition that addresses the foregoing problems of the prior art.

In one embodiment, the present invention relates to an adhesive paste applicable to skin in paste form comprising a mixture of: (a) a continuous phase comprising at least one styrene-containing thermoplastic elastomer, at least one compatible liquid rubber, polyisobutylene, and at least one oil; and (b) a discontinuous phase comprising at least one water soluble and/or water swellable polymer; wherein the adhesive paste contains about 25% to about 45% by weight, based on the total weight of the adhesive paste, of the mineral oil. In one embodiment, the oil comprises mineral oil.

In one embodiment, the present invention relates to an adhesive paste composition comprising a styrene-containing block copolymer pressure sensitive adhesive, present in a range from about 1 wt. % to about 9 wt. % of the composition; a polyisobutylene, present from about 15 wt. % to about 40 wt. % of the composition; and a mineral oil component, present from about 25 wt. % to about 45 wt. % of the composition, and at least one water-soluble and/or water-swellable absorbent polymer, present in an amount from about 25 wt. % to about 55 wt. % of the composition.

The fluid absorbing adhesive paste composition, in addition to the foregoing ingredients, may include additional absorbing materials, pharmaceutical agents, therapeutic agents, moisturizing agents, or other agents that assist in the healing process.

The present invention relates to improved pressure-sensitive adhesive hydrocolloid compositions that can be used, inter alia, to fill uneven folds of skin or scar tissue, and for molding around anatomical structures such as facial structures, in order to make a smooth platform for the adherence of a protective skin barrier. In particular, this invention relates to new fluid absorbing pressure-sensitive adhesive paste compositions that are significantly gentler to the skin than the prior art pastes. Specifically, this invention relates to pressure-sensitive adhesive hydrocolloid compositions that can be dispensed from tubes, which contain no alcohol diluents associated with prior art paste products, and that can be used in wound care dressings as well as in ostomy care.

The invention further relates to a tube dispensable pressure-sensitive adhesive hydrocolloid paste composition that is suitable for use on exuding wounds as an adjunct absorbent of wound exudate, and that is easily removable from the wound bed after it becomes saturated with wound exudate. The adhesive paste composition of the present invention may be employed as an adjunct product for use, for example, with conventional hydrocolloid dressings and may be used at any stage in the wound healing process.

The invention further relates to a tube dispensable, fluid absorbing, pressure-sensitive adhesive paste composition that can be sterilized by, for example, gamma radiation, in order that the aforesaid open wound contact use is safe.

### DETAILED DESCRIPTION

The present inventor has surprisingly found that the incorporation of a very small amount of a permanently tacky pressure-sensitive adhesive, into a composition otherwise containing polyisobutylene, oil and absorbent fillers, creates an adhesive fluid absorbing paste composition that is capable of being dispensed from a tube at room temperature. As used herein, the term “moldable” means that the paste will conform to the underlying irregular surface, i.e., the contours of the face and body. The moldable composition has sufficient cohesive strength to be able to be removed from the underlying surface as an integrated unit, without leaving residue. The adhesive paste is not overly sticky, so as not to be unmanageable. As used herein, “dispensed from a tube” means that the material to be dispensed may be dispensed by application of hand pressure by an adult person to the tube, where the tube has an orifice having an area of about 0.7 cm² or greater. In one embodiment, the composition has a rheology wherein it is hand-dispensable from a tube at a temperature in the range from about 0° C. to about 40° C.

The fluid absorbing adhesive paste comprises a continuous adhesive phase and a discontinuous absorbing phase dispersed in the continuous phase. The continuous phase comprises a mixture of a permanently tacky pressure sensitive adhesive containing at least one styrene-containing thermoplastic elastomer and at least one compatible liquid rubber; polyisobutylene and at least one oil. In one embodiment the oil comprises mineral oil. The discontinuous phase comprises at least one water soluble and/or water swellable absorbent polymer. The adhesive paste contains about 25% to about 45% by weight, based on the total weight of the adhesive paste, of the oil.

In one embodiment, the permanently tacky pressure-sensitive adhesive comprises a hot melt adhesive. In one embodiment, the permanently tacky pressure sensitive adhesive comprises a styrene-diolefin block copolymer together with a liquid rubber component. This adhesive may be combined with a polyisobutylene and an oil such as mineral oil, to provide the adhesive component (continuous phase) of the paste adhesive of the present invention. The adhesive component is combined with the water-soluble and/or water-swellable polymer component (discontinuous phase) to yield the paste adhesive of the present invention.

Thus, a fluid absorbing pressure-sensitive adhesive composition according to the invention comprises a mixture of a permanently tacky pressure-sensitive adhesive, a low molecular weight polyisobutylene and mineral oil, mixed together with at least one water soluble and/or water swellable absorbent polymer. In one embodiment, the permanently tacky pressure-sensitive adhesive comprises a styrene-containing block copolymer.

The paste adhesive composition of the present invention includes, in one embodiment, an adhesive component including at least one styrene-containing thermoplastic elastomer; at least one compatible liquid rubber; a polyisobutylene; and at least one oil. This adhesive component is combined or mixed with at least one water-soluble and/or water-swellable absorbent polymer to form the pressure-sensitive adhesive paste composition. In one embodiment, the adhesive composition of the present invention includes a permanently tacky pressure-sensitive adhesive component comprising a styrene-containing block copolymer and a liquid rubber; a low molecular weight polyisobutylene; and mineral oil.

In one embodiment, the adhesive component includes a styrene-containing thermoplastic elastomer and a liquid rubber. In one embodiment, the styrene-containing thermoplastic elastomer comprises a block or radial copolymer and a liquid rubber. In one embodiment, the permanently tacky pressure-sensitive adhesive is present in a range from about 1 wt. % to about 9 wt. % of the pressure-sensitive adhesive hydrocolloid paste composition.

Suitable styrene-containing permanently tacky pressure-sensitive adhesives useful in this embodiment include a styrenic component and a liquid rubber component. In one embodiment, use of the liquid rubber component with the styrenic component obviates the need for a conventional tackifier.

The styrenic components may include block or radial copolymers based on styrene-butadiene, styrene-isoprene or styrene ethylene-butylene. In addition, a low styrene synthetic copolymer of butadiene and styrene, commonly called SBR rubber, can be used as the thermoplastic elastomer. The elastomer may comprise linear or radial A-B-A block copolymers or mixtures of these A-B-A copolymers with simple A-B block copolymers. In one embodiment, the proportion of A-B block copolymers in the mixture of A-B-A and A-B block copolymers does not exceed about 85% by weight, and in other embodiments, lower percentages are used. In one embodiment, the proportion of A-B block copolymers in the mixture of A-B-A and A-B block copolymers does not exceed about 65% by weight, in another embodiment the proportion of A-B block copolymers in the mixture of A-B-A and A-B block copolymers does not exceed about 50% by weight, and in another embodiment, the proportion of A-B block copolymers in the mixture of A-B-A and A-B block copolymers does not exceed about 35% by weight, and in yet another embodiment, the proportion of A-B block copolymers in the mixture of A-B-A and A-B block copolymers does not exceed about 20% by weight. Here and elsewhere in the specification and claims, the limits of the ranges and ratios may be combined.

In one embodiment, the elastomeric component comprises linear or radial A-B-A block copolymers or mixtures of these linear or radial A-B-A block copolymers with simple A-B block copolymers. In these block copolymers the A-blocks are derived from styrene or styrene homologs and the B-blocks are derived from conjugated dienes or lower alkenes.

The A-B-A block copolymers are of the type which consist of A blocks derived from styrene or one of its homologs and B blocks derived from conjugated dienes, such as butadiene or isoprene, or from lower alkenes such as ethylene or butylene. The radial A-B-A polymers useful in this embodiment are of the type described for example in U.S. Pat. No.3,281,383 and conform to the general formula (A-B)ₙX, where A and B comprise blocks derived from the monomers described above in connection with the A-B-A copolymers, X is an organic or inorganic connecting moiety having a functionality of at least 2, and n is equal to the functionality of X. Homologs of styrene may include any known homolog of styrene for use in such A-B-A, A-B or (A-B)ₙX copolymers. For example, (C₁-C₈) alkylstyrenes such as a-methylstyrene, o-, m- and p-methylstyrenes, p-ethylstyrene, 2,4-dimethylstyrene, p-n-butylstyrene, p-t-butylstyrene, p-n-hexylstyrene, p-n-octylstyrene, p-n-nonylstyrene and p-n-decylstyrene), arylstyrenes (such as p-phenylstyrene), alkoxy-substituted styrenes (such as p-methoxystyrene), hydroxyl-substituted styrenes (such as p-hydroxystyrene), halogen-substituted styrenes (such as p-chlorostyrene and 3,4-dichlorostyrene) and mixtures of two or more of these (such as mixtures of styrene with at least one substituted styrenes) may be suitable styrene homolog. As used in the following, “styrene” may include homologs thereof.

Suitable styrene-diene copolymers are exemplified by a blend of linear styrene-isoprene-styrene triblock copolymer and linear styrene-isoprene diblock copolymer. Such a material is available from Kraton Polymers, Houston, Tex. as KRATON® D-1161 N (referred to as D-1 161 in Europe) and has a bound styrene content of about 15% and a diblock content of 17%. A second example is a blend of linear styrene-isoprene-styrene triblock copolymer and linear styrene-isoprene diblock copolymer available from Shell Chemical as KRATON® D-1 117P and which has a bound styrene content of about 17% and a diblock content of 33%.

An example of a suitable hydrogenated styrene-diene copolymer is a thermoplastic elastomer comprising a blend of linear triblock and diblock copolymer based on styrene and ethylene-butylene with a bound styrene of about 14 wt. %. Such a material is commercially available from Kraton Polymers as KRATON® G-1657M, which has a bound styrene content of about 13 wt. %. Another example is KRATON® G-1652 (referred to as G-1652E in Europe) from Kraton Polymers, which is a thermoplastic elastomer comprised of a linear triblock copolymer based on styrene and ethylene-butylene, S-E/B-S, with a bound styrene content of about 30 wt. %. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material is available from Kraton Polymers as KRATON® Research Product RP6919. This material has a styrene content of 18 wt. %, an isoprene content of 36 wt. % and an ethylene-butylene content of 46 wt. %.

In one embodiment, the permanently tacky pressure-sensitive adhesive component is comprised of the thermoplastic elastomer and the liquid rubber component, and is substantially resin-free.

Liquid rubbers useful in this embodiment are synthetic liquid isoprene rubber, depolymerised natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer. In one embodiment, the liquid rubbers have a molecular weight in a range from about 2500 to about 50,000. In one embodiment, the liquid rubbers have a glass transition temperature of less than about −50° C., and a melt viscosity at 38° C. in the range from about 500 to about 10,000 poises. It will be appreciated that other liquid rubbers known in the art could be useful in this embodiment of the present invention.

In one embodiment, the thermoplastic elastomer is a block copolymer of styrene and isoprene having a styrene content of about 13 wt. % and an isoprene content of about 87 wt. %, a glass transition temperature of about −60° C., a melt viscosity of about 2400 poises at 50° C. and has a weight average molecular weight of about 30,000 to about 50,000. KRATON® LVSI-101 is a material having such properties.

Another example of a useful liquid rubber is a liquid polyisoprene obtained by selectively or partially degrading a high molecular weight polyisoprene. An example of a commercially available partially degraded high molecular weight polyisoprene is ISOLENE® D-400 from Elementis Performance Polymers, Belleville, N.J., and this liquid rubber has a molecular weight of about 20,000. Other liquid rubbers which may be incorporated into the adhesive mixture include liquid styrene-butadiene rubbers, liquid butadiene rubbers, ethylene-propylene rubbers, etc., as noted above.

In one embodiment, the liquid rubber component comprises a low molecular weight liquid rubber. In one embodiment, the liquid rubber component has a number average molecular weight less than about 3,000.

In one embodiment, this permanently tacky pressure-sensitive adhesive is formed of a weight ratio of styrene-containing block copolymer to liquid rubber of about 1:0.25 to about 1:10, and in one embodiment from about 1:0.5 to about 1:7, and in another embodiment, from about 1:1.5 to about 1:3.5.

In one embodiment, the amount of liquid rubber corresponds to about 8 wt. % of the combination of the styrene-containing thermoplastic elastomer and the liquid rubber. In one embodiment, the maximum amount of liquid rubber is limited to about 5 wt. % of the combination of the styrene-containing thermoplastic elastomer and the liquid rubber.

In one embodiment, the amount of thermoplastic elastomer used in the adhesive paste composition includes amounts such that moldability and tube dispensability are not compromised. In one embodiment, the composition contains at least about 1 wt. % of the thermoplastic elastomer. However, if too much thermoplastic elastomer is present in the formulation, the compositions will be too elastic to function as a paste material or to be dispensed by hand from a tube. In one embodiment, the upper limit of thermoplastic elastomer in the pressure-sensitive adhesive hydrocolloid composition is about 5 wt. %, in one embodiment it is less than 5 wt. %, in another embodiment about 3 wt. %, and in yet another embodiment, about 2 wt. %.

The content of the permanently tacky pressure-sensitive adhesive component ranges from about 1 wt. % to about 9 wt. % of the composition. In one embodiment, the content of the permanently tacky pressure-sensitive adhesive component ranges from about 1.5 wt. % to about 5 wt. % of the composition.

A processing stabilizerofthe IRGANOX® 1010 described below may be used to protect the thermoplastic elastomer from excessive degradation during processing.

The adhesive paste composition of the present invention further includes a polyisobutylene component. In one embodiment, the polyisobutylene component is a low molecular weight polyisobutylene. In one embodiment, the polyisobutylene component is exemplified by the VISTANEX® LM series of polyisobutylenes, available from ExxonMobil Chemical Corporation. In one embodiment, the polyisobutylene has a Flory viscosity average molecular weight in the range from about 25,000 to about 75,000, and in one embodiment, from about 35,000 to about 70,000, and in one from about 40,000 to about 55,000. In one embodiment, the polyisobutylene has a Brookfield viscosity at 175° C. within the range from about 10,000 to about 170,000 mpa.sec, and in one embodiment, from about 20,000 to about 140,000 mpa.sec, and in another from about 25,000 to about 70,000 mpa.sec. Brookfield viscosity is determined by measuring the shearing stress on a spindle rotating at a definite, constant speed while it is immersed in the sample. Brookfield viscosity is measured in centipoises or mpa.sec. Viscosity is a function of shear rate and is defined as shear stress/shear rate, and is measured according to ASTM D3236. In one embodiment, the Staudinger molecular weight of the low molecular weight polyisobutylenes ranges from about 5,000 to about 20,000, and in another embodiment, the Staudinger molecular weight ranges from about 10,000 to about 12,000.

In one embodiment, the low molecular weight polyisobutylene component is present in the pressure-sensitive adhesive hydrocolloid composition at between about 15 wt. % and about 40 wt. % of the total formulation, and in one embodiment, between about 20 wt. % and about 30 wt. % of the total formulation.

The adhesive paste composition further includes an oil component. In one embodiment, the oil component comprises mineral oil. In one embodiment, a portion of the mineral oil may be replaced by a liquid hydrocarbon or liquid polymeric material or by a natural vegetable oil. In one embodiment, the mineral oil is exemplified by the Kaydol series of materials from Witco Chemical. Witco White Mineral Oil USP has a viscosity at 40° C. between about 60 and about 75 mm²/s, and in one embodiment, between about 63 and about 70 mm²/s, as determined by test method ASTM D-445. However, any suitable mineral oil may be used. Mineral oil may also be referred to as liquid petrolatum, mineral spirits, adepsine oil, alboline, glymol, liquid paraffin, paraffin oil or saxol, some of which may be used as trade names. Mineral oil has a boiling point in the range from about 179° C. to about 210° C.

In one embodiment, the oil component may comprise corn oil soybean oil, cottonseed oil, castor bean oil, palm oil, coconut oil, sunflower seed oil, canola oil, other known vegetable oils, animal oils such as fish oil, lard and tallow, and may further comprise synthetic oils triglycerides.

In one embodiment, the mineral oil component is present in the adhesive paste composition within the range of about 10 wt. % to about 45 wt. %, and in one embodiment, from about 15 wt. % to about 40 wt. %.

In one embodiment a portion of the mineral oil component may be replaced by a liquid hydrocarbon or a liquid polymeric component, as defined above. In one such embodiment, at least about 25 wt. % of the composition is mineral oil, and the additional liquid hydrocarbon or liquid polymer component is added thereto, up to a total limit of about 45 wt. % for the combined mineral oil and liquid hydrocarbon or liquid polymeric component.

In one embodiment, the adhesive paste composition further comprises low molecular weight polybutene. The low molecular weight polybutene components are exemplified by the HYVIS® series of materials from BP, and by the PARAPOL® series of products from Exxon Chemical Corporation.

In one embodiment, a suitable processing stabilizer may also be included in the pressure-sensitive adhesive composition. Suitable stabilizers include those indicated for use with styrene-olefin-styrene block copolymer thermoplastic elastomers such as organophosphites and the so-called hindered phenols, but any suitable stabilizers may be employed. An example of an organophosphite stabilizer is tris(nonylphenyl)phosphite, available as POLYGARD® HR, manufactured by Uniroyal.

Particularly useful stabilizers are the hindered phenols, IRGANOX® 1010 and IRGANOX® 565, manufactured by Ciba. IRGANOX® 1010 is pentaerythritol tetrakis (3-(3,5-di-tert-butyl-4hydroxyphenyl) propionate. IRGANOX® 565 is 2,6-di-tert-butyl4-(4,6-bis (octylthio)-1,3,5-triazin-2yl-amino)phenol. Stabilizers may be used separately or in combination, and suitable ranges are within about 0.1% to about 1.5%, or in one embodiment, from about 0.3% to about 1%, by weight based on the weight of the combined styrene-containing thermoplastic elastomer and liquid rubber. When present, the stabilizers are added to the adhesive component, as is shown in the examples.

Additional ingredients such as tackifiers and plasticisers may be added to the pressure-sensitive adhesive composition, to modify tack and optimize adhesion properties. However, tackifiers used in prior art hydrocolloid adhesives may make the present composition too sticky. Thus, in one embodiment, the pressure-sensitive adhesive composition is substantially free of any added tackifier. As used herein, the term “tackifier” or “tackifying resin” includes: (a) natural and modified rosins such, for example, as gum rosin, wood rosin, tall-oil rosin, distilled rosin, hydrogenated rosin, dimerized rosin, and polymerized rosin; (b) glycerol and pentaerythritol esters of natural and modified rosins, such, for example, as in the gycerol ester of pale wood rosin, the glycerol ester of hydrogenated rosin, the glycerol ester of polymerized rosin, the pentaerythritol ester of hydrogenated rosin, and the phenolic modified pentaerythritol ester of rosin; polyterpene resins having a softening point, as determined by ASTM method E28 58T, of from about 60° to 140° C., the latter polyterpene resins generally resulting from the polymerization of terpene hydrocarbons, such as the bicyclic mono-terpene known as pinene, in the presence of Friedel-Crafts catalysts at moderately low temperatures; (d) phenolic-modified terpene resins such, for example, as the resin product resulting from the condensation in an acidic medium, of a bicyclic terpene and a phenol; and (e) aliphatic petroleum hydrocarbon resins having a Ball and Ring softening point of from about 60° to 140° C., the latter resins resulting from the polymerization of monomers consisting primarily of olefins and diolefins. Thus, the low molecular weight polyisobutylenes disclosed for use herein are not included within the definition of tackifier as used herein. In addition, tackifying resins are usually solids at ordinary temperatures, while the low molecular weight polyisobutylenes disclosed herein are liquids at ordinary temperatures

Optional fillers such as silica and pigments and optional active ingredients such as antimicrobial compounds may also be incorporated into the pressure-sensitive adhesive paste compositions of this embodiment of the invention. Silver sulfadiazine and benzalkonium chloride represent non-limiting examples of such antimicrobial ingredients.

In one embodiment, the pressure-sensitive adhesive composition is substantially free of added wax, mineral wax or petroleum jelly, and in one embodiment is substantially free of microcrystalline wax.

In one embodiment, the pressure-sensitive adhesive paste composition is substantially free of copolymers such as ethylene vinyl acetate, and in one embodiment the composition is substantially free of copolymers of ethylene vinyl alcohol.

In one embodiment, the pressure-sensitive adhesive composition is substantially free of additives such as aloe, aloe vera extract, etc.

In one embodiment, the pressure-sensitive adhesive composition is substantially free of absorbent additives such as silica, aerosil, diatomaceous earth, zeolites or molecular sieve.

In one embodiment, the pressure-sensitive adhesive paste composition is substantially free of organic solvent, particularly alcohol.

In one embodiment, the pressure-sensitive adhesive paste composition is substantially resin-free.

In one embodiment, the pressure-sensitive adhesive paste composition is substantially free of a tackifier.

In one embodiment, the pressure-sensitive adhesive composition is substantially free of radiation cross-linked polymers.

The adhesive paste composition includes a discontinuous phase comprising at least one water-soluble and/or water-swellable polymer as a fluid absorbing component. In one embodiment the polymer comprises a hydrophilic polymer that is soluble or insoluble but swellable in water as the fluid absorbing component. At least one swellable polymer may be present in the composition. Suitable insoluble swellable polymers include cross-linked sodium carboxymethyl cellulose, crystalline sodium carboxymethyl cellulose, cross-linked dextran and starch-acrylonitrile graft copolymer. The swellable polymer may also include a so-called “super absorbent” material such as starch sodium polyacrylate. Other hydratable polymers such as gluten and polymers of methyl vinyl ether and maleic acid and derivatives thereof may also be included in the water-soluble and/or water-swellable polymer. Suitable water soluble polymers include sodium carboxymethyl cellulose, pectin, gelatine, guar gum, locust bean gum, collagen, karaya gum and starch, particularly maize starch, and the like.

In one embodiment, the hydrophilic absorbent polymers comprise at least one of alginic acid, sodium alginate, calcium alginate, cellulose-derived material, starch or a modified starch, a copolymer of a starch or a cellulosic material, a water soluble hydrocolloid, a synthetic resin, a mannan, seaweeds, a plant mucilage, or combinations of two or more thereof.

In one embodiment, the water-soluble and/or water-swellable polymer comprises about 25% to about 55% by weight of the total weight of the pressure-sensitive adhesive paste composition. In one embodiment, the water-soluble and/or water-swellable polymer constitutes about 30 to about 50% by weight of the pressure-sensitive adhesive paste composition, and in another embodiment, the water-soluble and/or water-swellable polymer constitutes from about 35 to about 45% by weight of the pressure-sensitive adhesive paste composition.

The water-soluble and/or water-swellable polymer may be comprised of any combination of soluble and/or insoluble absorbents. In one embodiment, the water-soluble and/or water-swellable polymer may comprise at least one hydrophilic absorbent polymer that is soluble or which absorbs and/or is swellable in water. In one embodiment, the hydrophilic absorbent polymer includes alginic acid, sodium alginate, calcium alginate, cellulose-derived material, starch or a modified starch, a copolymer of a starch or a cellulosic material, a water soluble hydrocolloid, a synthetic resin, a mannan, seaweeds, and a plant mucilage or combinations of two or more thereof. At least one such hydrophilic absorbent polymer may be present and a mixture of soluble and insoluble hydrophilic absorbent polymers can be used. Suitable swellable hydrophilic absorbent polymers include, e.g., cross-linked sodium carboxymethyl cellulose, crystalline sodium carboxymethyl cellulose, cross-linked dextran and starch-acrylonitrile graft copolymer. The swellable polymer may also be a so-called “super absorbent” material such as starch sodium polyacrylate. In one embodiment, the swellable polymer is other than a “super absorbent” material. Other hydrophilic absorbent polymers such as gluten and polymers of methyl vinyl ether and maleic acid and derivatives thereof may also be included in the water-soluble and/or water-swellable polymer.

In one embodiment, the water-soluble and/or water-swellable polymer may comprise at least one water-soluble hydrocolloid, alone or blended with at least one swellable polymer. Such soluble hydrocolloids include naturally derived products such as pectin, gelatin, starches, guar gum, locust bean gum, gum arabic, gum karaya, collagen, karaya gum, alginic acid and its sodium and/or calcium salts. Also useful are the synthetic hydrocolloids such as sodium carboxymethyl cellulose, crosslinked sodium carboxymethyl cellulose, polyvinyl alcohol, polyvinyl pyrrolidone, high molecular weight polyethylene glycols and polypropylene glycols.

In one embodiment, the water-soluble and/or water-swellable polymer may comprise at least one of starches such as flour starch, corn starch, potato starch, etc. In another embodiment, the water-soluble and/or water-swellable polymer may comprise mannan, such as yeast gum, manna or konjak. In another embodiment, the water-soluble and/or water-swellable polymer may comprise at least one of various seaweeds such as agar-agar, sodium alginate, etc. In another embodiment, the water-soluble and/or water-swellable polymer may comprise at least one plant mucilage such as tragacanth gum, gum arabic, karaya gum, guar gum, psyllium seed gum, dammar gum, pectin etc., various proteins such as gelatin, collagen, casein, etc. In another embodiment, the water-soluble and/or water-swellable polymer may comprise at least one cellulose-derived material such as carboxymethyl cellulose, hydroxyethyl cellulose, methyl cellulose, etc., modified starches such as soluble starch, carboxymethyl starch, dialdehyde starch, a cross-linked dextrin, etc. In another embodiment, the water-soluble and/or water-swellable polymer may comprise at least one copolymer of starch or cellulose, such as starch-acrylonitrile graft copolymer, a starch polyacrylate salt. In another embodiment, the water-soluble and/or water-swellable polymer may comprise at least one synthetic resin such as polyvinyl alcohol, sodium polyacrylate, polyethylene oxide, etc., and copolymers of starches or celluloses and acrylonitrile, acrylic acid, methacrylic acid, vinyl alcohol, vinyl chloride, etc. In one embodiment, the water-soluble and/or water-swellable polymer may comprise at least one of plant mucilage such as tragacanth gum, gum arabic, karaya gum, guar gum, psyllium seed gum, dammar gum, pectin, etc., the celluloses such as CMC (carboxymethyl cellulose), HEC (hydroxyethl cellulose), etc., and the copolymers of starches or celluloses and acrylonitrile, acrylic acid, sulfuric acid, vinyl sulfonate, etc. The foregoing embodiments may be combined with one another, and may be combined with water swellable polymers and/or super-absorbent materials.

In one embodiment, the water swellable polymers include, for example, hydroxypropylcellulose (HPC) and polyethylene oxide (PEO). HPC is available from commercial suppliers including, for example, Aqualon, Inc., (Wilmington, Del.). The useful HPC generally has an average molecular weight in the range of about 60,000 to 1,200,000. In another embodiment, the water swellable polymer includes homopolymers and copolymers of carboxymethyl cellulose, hydroxyethyl cellulose, hydroxymethyl cellulose). In another embodiment, the water swellable polymer includes a water-soluble or water-swellable polymer derived from acrylic acid or a pharmaceutically acceptable salt thereof, such as the polyacrylic acid polymers as follows: Polycarbophil (Noveon AA-1), carbomer (Carbopol 974P or 971 P or 907), or a water-soluble salt of a co-polymer of methyl vinyl ether and maleic acid or anhydride (Gantrez MS-955).

### EXAMPLES

### Example A

In one embodiment, the adhesive paste compositions of the invention may be prepared as follows. The thermoplastic elastomer and the liquid rubber are blended together in a suitable mixer, normally a sigma blade mixer with an extruder discharge. The mixer is heated to about 170° C. A nitrogen flow of about 60 ml/sec through the mixer reduces the possibility of oxidative degradation of the elastomer during processing. About 1% phrof a suitable stabilizer, forexample, IRGANOX® 1010, can be added at this stage. The thermoplastic elastomer is allowed first to blend in the mixer until it coalesces. Normally a small amount of the liquid rubber, say 10-20% of the total amount to be added, is added to the whole amount of the thermoplastic elastomer and the liquid rubber is allowed to blend with the softened thermoplastic elastomer. When all this 10-20% of the liquid rubber oil has been absorbed, another portion of the liquid rubber is added, for example about 20-30%, and the liquid rubber is absorbed into the styrene-olefin-styrene rubber. This is continued until all the liquid rubber is added, when a pourable tacky intermediate adhesive is obtained. The mixer blades are stopped, the direction of the screw is reversed, and the intermediate adhesive is removed from the mixer. It is run off into suitably release coated containers and allowed to cool.

The mixer is cleaned, stabilized at 90° C. and the powdery hydrocolloid ingredients are charged to the mixer together with the low molecular weight polyisobutylene and the mineral oil. These are blended until uniform for about 10 minutes and then the previously blended permanently tacky pressure-sensitive adhesive component is added and the mixer temperature is raised to 105° C. After mixing at 105° C. for 15 minutes the temperature of the mixer is dropped to about 80° C. and any other additives, such as the low molecular weight polybutene is added (in various embodiments as appropriate). Mixing is continued normally for a further approximately 20 minutes. The fully mixed mass is then removed from the mixer, and is filled into suitable containers, such as individual tubes. Alternatively, it can be stored in a larger container and later be dispensed into the individual tubes. Tubes suitable for use with mineral oil, having an adhesive coated band at the closeable end, or other suitable sealing means, are recommended to avoid any leakage.

The above manufacturing process has been described with respect to a two-step process in which the permanently tacky pressure-sensitive adhesive comprising the styrene-containing thermoplastic elastomer, liquid rubber and stabilizer is first manufactured and isolated and which is then subsequently used to make the compositions of this embodiment of the invention. It will be appreciated by anyone of ordinary skill in the art that no process limitation is implied and the adhesives of the invention may equally well be prepared by other methods, such as in a one-pot process, with no isolation of the intermediate permanently tacky pressure-sensitive adhesive.

Assessment of the moldability of the pressure-sensitive adhesive paste compositions may be made using the test methods described in Adhesives Age, September 1997, pp 18-23. The equipment used is a probe tester manufactured by Stable Micro Systems, Godalming, Surrey, England, driven by custom designed software. Using the equipment, the energy absorbed by the adhesive as the probe penetrated the adhesive is determined by a transducer. The probe is moved up and down by a rotating screw which is driven by a stepping motor. The displacement of the probe is measured through the motor rotation. For each measurement, the probe is a stainless steel ball of 25.4 mm in diameter. The compressive force is 4.5N and the test speed is 0.04 mm/sec.

### Examples 1-11

The invention is further described by the following non-limiting Examples.

The raw materials used in the examples are as follows:

LVSI-101 - Kraton Polymers: Styrene-isoprene-styrene diblock copolymer (SI) liquid rubber.

KRATON® D-1161NS—Kraton Polymers: SIS thermoplastic elastomer

IRGANOX® 1010—Ciba Chemicals: Stabilizer

VISTANEX® LMMS—Exxon Chemical; low molecular weight polyisobutylene

Kaydol Mineral Oil—Witco Chemical

GENU Pectin USP100—Hercules Chemical

GENU Pectin LM-104 AS-FS—Hercules Chemical

Blanose 7H4XF—Hercules Chemical

Natrasol 250 CM Plus—Hercules Chemical

Natrasol Plus CS, Grade 330—Hercules Chemical

Carbopol Ultrez 10NF—Noveon Inc.

Calcium Alginate—Kelco

### Example 1

This example illustrates preparation of the intermediate permanently tacky pressure-sensitive adhesive component of the adhesive paste of the present invention.

| Component | wt. % | Amount, gm |
|---|---|---|
| LVSI-101 | 79.37 | 400 |
| KRATON ® D-1161N | 19.84 | 100 |
| IRGANOX ® 1010 | 0.79 | 4 |
| TOTAL | 100.00 | 504 |

A Z-blade mixer of 1 kg capacity is purged with nitrogen gas and heated to 160° C. The speed of the front, faster, blade is 30 rpm. The KRATON® D-1161K and the IRGANOX® 1010 are charged to the mixer at 160° C., and the mixer is started. After mixing for 5 minutes, when the rubbery crumb coalesces, 50 gm of the LVSI-101 is added with continued mixing and nitrogen purging. After a further ten minutes, the temperature is raised to 170° C. and the mixer front blade speed increased to 47 rpm. The LVSI had at this point should be completely mixed with the rubber, and a further 51 gm of LVSI is added. Ten minutes later, after blending of the second portion of the LVSI, a further 48 gm of LVSI is added, and is mixed for a further 10 minutes. In this way, approximately 50 gm portions of the charge of LVSI are added every 10 minutes until all the 400 gm has been added. After mixing for another 15 minutes, the intermediate adhesive is dumped from the mixer. The total time for this operation is about 90 minutes.

The above preparation or compounding process has been described with respect to a two-step process in which the hot melt adhesive comprising the styrene-containing thermoplastic elastomer and the liquid rubber and the stabilizer is first manufactured and isolated and which is then subsequently used to make the compositions of this embodiment of the invention. It will be appreciated by anyone of ordinary skill in the art that no process limitation is implied and the adhesives of the invention may equally well be prepared by other methods, such as in a one-pot process, with no isolation of the intermediate hot melt adhesive.

### Examples 2-11

The following examples illustrate preparations of the pressure-sensitive adhesive composition of the present invention. In the following examples, the water soluble and/or water swellable absorbent polymer ingredients, the PIB and the mineral oil are mixed as described above, and the permanently tacky pressure-sensitive adhesive prepared in Example 1 is combined with these ingredients as described above.

| | Ex. 2 | Ex. 3 | Ex. 4 | Ex. 5 | Ex. 6 | Ex. 7 | Ex. 8 | Ex. 9 | Ex. 10 | Ex. 11 |
|---|---|---|---|---|---|---|---|---|---|---|
| Formulation | Wt. % | Wt. % | Wt. % | Wt. % | Wt. % | Wt. % | Wt. % | Wt. % | Wt. % | Wt. % |
| Ex. 1 | 9.00 | — | 3.32 | 5.54 | 1.66 | 1.66 | 1.66 | 1.66 | 1.66 | 4.96 |
| adhesive | | | | | | | | | | |
| VISTANEX ® | 20.00 | 23.07 | 23.07 | 23.07 | 23.07 | 23.07 | 23.07 | 23.07 | 23.07 | 22.06 |
| LMMS | | | | | | | | | | |
| Pectin | 16.00 | 14.77 | 13.66 | 12.92 | 14.21 | 14.21 | 8.53 | 8.53 | 8.53 | 8.33 |
| USP100 | | | | | | | | | | |
| Natrasol | — | — | — | — | — | 7.11 | 8.53 | — | — | — |
| 2500CM | | | | | | | | | | |
| Plus | | | | | | | | | | |
| Natrasol | — | — | — | — | — | — | — | 8.53 | 8.53 | 8.33 |
| Plus CS, | | | | | | | | | | |
| Grade 330 | | | | | | | | | | |
| Blanose | 16.00 | 14.77 | 13.66 | 12.92 | 7.11 | — | 8.53 | 8.53 | 8.53 | 8.33 |
| 7H4XF | | | | | | | | | | |
| GENU | — | 14.77 | 13.66 | 12.92 | 14.21 | 14.21 | 8.53 | 8.53 | 8.53 | 8.33 |
| Pectin LM- | | | | | | | | | | |
| 104 AS-FS | | | | | | | | | | |
| Carbopol | — | — | — | — | — | — | — | — | 8.53 | 8.33 |
| Ultrez 10NF | | | | | | | | | | |
| Calcium | — | — | — | — | 7.11 | 7.11 | 8.53 | 8.53 | — | — |
| Alginate | | | | | | | | | | |
| Mineral Oil | 39.00 | 32.63 | 32.63 | 32.63 | 32.63 | 32.63 | 32.63 | 32.63 | 32.63 | 32.33 |
| Total | 100.0 | 100.01 | 100.00 | 100.00 | 100.00 | 100.00 | 100.01 | 100.01 | 100.01 | 100.00 |

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. An adhesive paste applicable to skin in paste form comprising a mixture of:
a continuous phase comprising at least one styrene-containing thermoplastic elastomer, at least one compatible liquid rubber, polyisobutylene, and at least one oil; and
a discontinuous phase comprising at least one water soluble and/or water swellable absorbent polymer;
wherein the adhesive paste contains about 25% to about 45% by weight, based on the total weight of the adhesive paste, of the oil.

2. The adhesive paste of claim 1 wherein the oil comprises mineral oil.

3. The adhesive paste of claim 1 wherein the paste comprises mineral oil in an amount within the range of about 30% to about 40% by weight, based on the total weight of the paste.

4. The adhesive paste of claim 1 wherein the weight ratio of the styrene-containing thermoplastic elastomer to liquid rubber is from about 1:0.5 to about 1:7.

5. The adhesive paste of claim 1 wherein discontinuous phase comprises at least one insoluble but water-absorbent and swellable polymer.

6. The adhesive paste of claim 5 wherein the water insoluble polymer comprises at least one of cross-linked sodium carboxymethyl cellulose, crystalline sodium carboxymethyl cellulose, cross-linked dextran, calcium alginate, starch-acrylonitrile graft polymer, starch sodium polyacrylate, gluten, polymers of methylvinyl ether and maleic acid and derivatives thereof.

7. The adhesive paste of claim 1 wherein the discontinuous phase comprises at least one water soluble hydrocolloid.

8. The adhesive paste of claim 7 wherein the soluble hydrocolloid comprises at least one of sodium carboxymethyl cellulose, pectin, gelatin, guar gum, locust bean gum, collagen, karaya gum, alginic acid, sodium alginates, sodium-calcium alginates, polyvinyl alcohol, polyvinyl pyrrolidone, polyethylene glycol and polypropylene glycol.

9. The adhesive paste of claim 1 wherein the solid rubber comprises at least one linear or radial A-B-A block copolymer based on styrene-butadiene, styrene isoprene or hydrogenated styrene-diene copolymers.

10. The adhesive paste of claim 9 wherein the continuous phase comprises up to 85% by weight relative to the A-B-A block copolymer(s) of one or more styrene-butadiene, styrene isoprene or hydrogenated styrene-diene AB block copolymers.

11. The adhesive paste of claim 1 wherein the liquid rubber has a molecular weight in the range of about 25,000 to about 50,000.

12. The adhesive paste of claim 1 wherein the liquid rubber has a glass transition temperature of less than −50° C.

13. The adhesive paste of claim 1 wherein the liquid rubber has a viscosity at 38° C. in the range of 500 to about 10,000 poises.

14. The adhesive paste of claim 1 wherein the liquid rubber comprises at least one of synthetic liquid isoprene rubber, depolymerized natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer.

15. The adhesive paste of claim 1 wherein the content of the polyisobutylene is from about 15% by weight to about 40% by weight of the total adhesive paste.

16. The adhesive paste of claim 1 wherein the polyisobutylene has a Flory viscosity average molecular weight in the range from about 25,000 to about 75,000.

17. The adhesive paste of claim 1 further comprising a polymer stabilizer.

18. The adhesive paste of claim 1 wherein the discontinuous phase comprises from about 25% by weight to about 55% by weight of the total weight of the adhesive paste.

19. The adhesive paste of claim 1 wherein the discontinuous phase comprises from about 30% by weight to about 40% by weight of the total weight of the adhesive paste.

20. The adhesive paste of claim 1 further comprising at least one low molecular weight polybutene.

21. The adhesive paste of claim 1 wherein the weight of the combination of the styrene-containing thermoplastic elastomer and the liquid rubber is the range of about 1% to about 9%, based on the total weight of the adhesive paste.

22. The adhesive paste of claim 1 wherein the paste is substantially free of a tackifier.

23. The adhesive paste of claim 1 wherein the composition is dispensable from a tube having an orifice of about 0.7 cm² and greater.

24. A wound dressing comprising an adhesive paste wherein the adhesive paste comprises:
a continuous phase comprising at least styrene-containing thermoplastic elastomer, at least one compatible liquid rubber, polyisobutylene, and mineral oil; and
a discontinuous phase comprising at least one water soluble and/or water swellable polymer;
wherein the adhesive paste contains about 25% to about 45% by weight, based on the total weight of the adhesive paste, of the mineral oil.

25. An adhesive paste applicable to skin in paste form comprising a mixture of:
a continuous phase comprising 0.1% to about 3.0% by weight of at least one styrene-containing thermoplastic elastomer, 0.5 % to about 9.0% by weight of at least one compatible liquid rubber, 15% to about 40% byweight of polyisobutylene, and 25% to about 45% by weight of mineral oil; and
a discontinuous phase comprising 25% to about 55% by weight of at least one water soluble and/or water swellable polymer.
